(19) United States
(12) Patent Application Publication
(10) Pub. No.: US 2021/0068677 A1
(43) Pub. Date: Mar. 11, 2021

(54) OPTICAL FLOW SIMULATORS AND METHODS OF USING THE SAME

(71) Applicant: MC10, Inc., Lexington, MA (US)

(72) Inventors: Milan Raj, Natick, MA (US); Evan Hovaniak, Wycoff, NJ (US); Bryan McGrane, Denver, CO (US); Adam Leech, Newton, MA (US)

(21) Appl. No.: 16/612,330

(22) PCT Filed: Apr. 30, 2018
(86) PCT No.: PCT/US2018/030139

§ 371 Date: Nov. 8, 2019

Related U.S. Application Data
(60) Provisional application No. 62/504,808, filed on May 11, 2017.

(51) Int. Cl. A61B 5/0295; A61B 5/00; A61B 5/1495; A61B 5/024

(52) U.S. Cl. CPC A61B 5/0295; A61B 5/7207; A61M 2205/3306; A61B 5/02416; A61B 5/1495

(57) ABSTRACT

An optical flow simulator includes a disk, a stepper motor, and a microcontroller. The disk has a color distribution varied along a path on the disk. The stepper motor is configured to rotate a spindle, and the spindle is coupled to the disk. The microcontroller controls the stepper motor such that the spindle and the disk rotate a predefined rotational velocity. The color distribution produces a simulated optical signal responsive to rotation of the disk and the predefined rotational speed.

### CROSS-REFERENCE TO RELATED APPLICATIONS

The present application claims the benefit of and priority to U.S. Provisional Patent Application No. 62/504,808, filed May 11, 2017, entitled, “Optical Flow Simulators and Methods of Using the Same,” which is incorporated by reference herein in its entirety.

### TECHNICAL FIELD

The present disclosure relates generally to devices and methods for simulating optical flow and, more particularly, to an optical flow simulator for simulating blood flow for testing an optical sensor.

### BACKGROUND

Optical blood flow measurement is widely used in a variety of clinical and consumer devices in the form of Photoplethysmography (“PPG”). Using a phototransmitter (e.g., a light-emitting diode) and a photodetector, a PPG sensor can be used to determine the movement of blood in a vein or artery near the surface of a subject's skin by measuring the amount of light absorbed by red hemoglobin in the blood. When developing and/or manufacturing PPG sensors, it is often necessary to validate the design of the sensor and verify proper functioning by testing the sensor on an individual. Testing PPG sensor design and function directly on individuals gives rise to testing efficiencies and other logistical problems. For example, in order to verify the PPG sensor's ability to detect unhealthy PPG patterns, the PPG sensor much be tested on an individual with unhealthy blood flow, thereby severely limiting the pool of potential test subjects. Further, other solutions for simulating blood flow involve complicated systems (e.g., pumping water through a tube with a peristaltic pump), and are often cost prohibitive. Thus, new devices and methods are needed for validating PPG sensor design and verifying sensor performance. The present disclosure addresses these and other problems.

### SUMMARY

In accordance with some embodiments of the invention, an optical flow simulator includes a stepper motor controlled by a microcontroller to move a surface having color distribution that varies along a path. In accordance with some embodiments of the invention, the colored (e.g., green, yellow, blue, purple, red) surface can take the form of a disk, a wheel or cylindrical surface, a belt, a spherical surface, or a conical surface. In accordance with some embodiments of the invention, the colored (e.g., green, yellow, blue, purple, red) surface can extend over the sides of polygonal structure, such as a square, a rectangle or any other n sided object. The microcontroller controls the speed of movement (e.g., rotation) of the colored surface to simulate the speed of the fluid flow. A sensor under test, such as a reflective PPG sensor, can be placed in proximity to the colored (e.g., green, yellow, blue, purple, red) surface such that it detects the change in color or amplitude of the light reflecting from the colored surface to simulate fluid flow under the sensor.

According to some implementations of the present disclosure, the optical flow simulator includes a disk coupled to a stepper motor that is connected to microcontroller configured to control the speed of rotation of the disk. The disk can include a predefined color distribution that is varied along a circular path or track on the disk such that when the disk rotates, the color along the circular path changes in a predefined way. The stepper motor includes a spindle that is coupled to the disk, either directly or indirectly (e.g., by gears, belts, or a transmission). The microcontroller controls the rotational speed of the stepper motor and causes the disk to rotate at a predefined rotational speed. Responsive to rotation of the disk at the predefined rotational speed, the color distribution on the surface of the disk produces a simulated optical signal that can be used to test a reflective PPG sensor, for example.

According to some implementations of the present disclosure, the optical flow simulator includes a belt coupled to a stepper motor that is connected to a microcontroller configured to control the speed of rotation of the belt. The belt can include a color distribution linearly varied along a path defined on a surface of the belt. The belt can be fitted over a plurality of rollers or pulleys. The stepper motor includes a spindle that is coupled to the belt, either directly or indirectly (e.g., by gears, belts, or a transmission). The microcontroller controls the rotational speed of the stepper motor and causes the belt to move at a predefined linear speed. Responsive to the movement of the belt at the predefined speed, the color distribution on the belt surface produces a simulated optical signal that can be used to test a reflective PPG sensor, for example.

According to some implementations of the present disclosure, an optical flow simulator includes a semi-translucent disk, belt or cylinder that moves (e.g. rotates). The semi-translucent disk includes a predefined light transmissivity distribution that is varied along a path. The stepper motor includes a spindle that is coupled to the semi-translucent disk, belt or cylinder, either directly or indirectly (e.g., by gears, belts, or a transmission). The microcontroller controls the rotational speed of the stepper motor and causes the spindle to rotate the disk, belt or cylinder at a predefined rotational speed. Responsive to the movement of the semi-translucent disk, belt or cylinder at the predefined speed, the light transmissivity distribution produces a simulated optical signal that can be used to test a transmissive PPG sensor, for example.

These and other aspects of the present invention will become more apparent from the following detailed description of the system in conjunction with the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings, which are incorporated into this specification, illustrate one or more exemplary embodiments of the inventions and, together with the detailed description, serve to explain the principles and applications of these inventions. The drawings and detailed description are illustrative, and are intended to facilitate an understanding of the inventions and their application without limiting the scope of the invention. The illustrative embodiments can be modified and adapted without departing from the spirit and scope of the inventions.

FIG. 1A is a side view of a reflective optical sensor according to some implementations of the present disclosure;

FIG. 1B is a side view of a transmissive optical sensor according to some implementations of the present disclosure;

FIG. 1C is a photoplethysmogram (PPG) waveform according to some implementations of the present disclosure;

FIG. 2 is a top view of an optical flow simulator according to some implementations of the present disclosure;

FIG. 3 is a perspective view of the optical flow simulator of FIG. 2;

FIG. 4A is top view of a disk of the optical flow simulator of FIG. 2;

FIG. 4B is a perspective view of a disks for use with optical flow simulator of FIG. 2;

FIG. 5 is a flow diagram of an algorithm for selecting a color distribution for the disk of FIG. 4A;

FIG. 6A is plot of a raw PPG signal;

FIG. 6B is a sample period of the raw PPG signal of FIG. 6A;

FIG. 6C is a plot of the raw PPG signal of FIG. 6A and a simulated optical signal produced by the optical flow simulator of FIG. 2;

FIG. 7 is a top view of an optical flow simulator according to some implementations of the present disclosure;

FIG. 8A is a top view of an optical flow simulator according to some implementations of the present disclosure; and

FIG. 8B is a side view of the optical flow simulator of FIG. 8A.

The present disclosure is susceptible to various modifications and alternative forms, and some representative embodiments have been shown by way of example in the drawings and will be described in detail herein. It should be understood, however, that the inventive aspects of the disclosure are not limited to the particular forms disclosed. Rather, the disclosure is to cover all modifications, equivalents, and alternatives falling within the spirit and scope of the invention as defined by the appended claims.

### DETAILED DESCRIPTION

The present invention is directed to methods and systems for testing and calibrating optical flow sensors by simulating the optical signals perceived by the sensor. In accordance with embodiments of the invention, the optical sensors detect either reflected or transmitted light signals (e.g., color and/or intensity) and can be used to detect fluid flow, for example, blood flow via PPG. In accordance with some embodiments of the invention, the system includes a surface that includes a predefined optically varying pattern that can be moved in proximity to the sensor to simulate the optically reflective or optically transmissive light signals that are received by the sensor. Using the predefined optical patterns, the optical sensors, such as PPG sensors can be calibrated and tested.

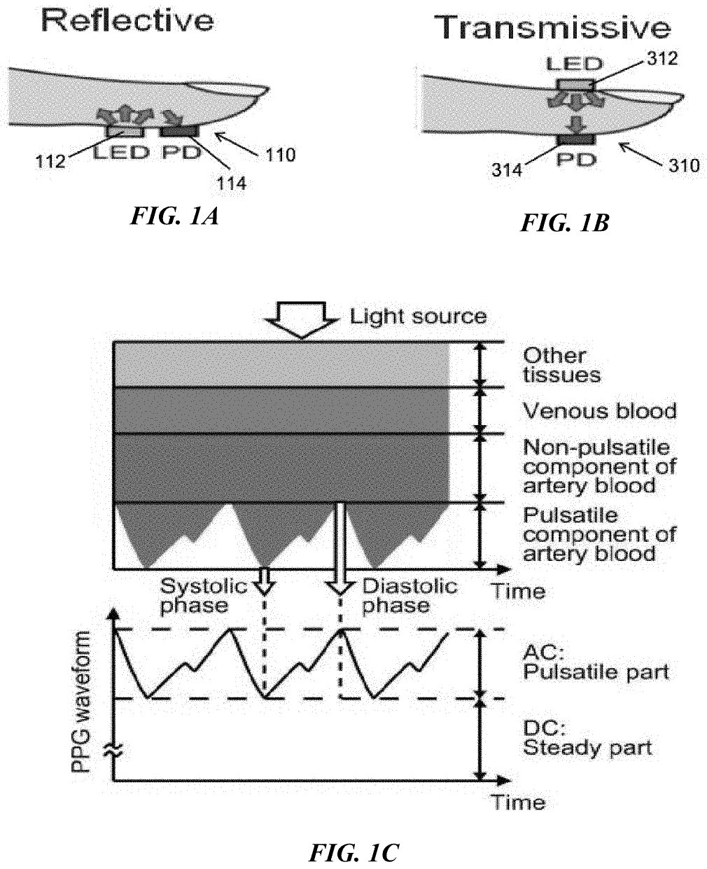

FIG. 1A shows the reflective optical sensor 110 that includes a photo transmitter or light source 112 (e.g., a light-emitting diode or “LED”) and a photodetector 114. The reflective optical sensor 110 can be used to obtain a photoplethysmogram (“PPG”) using a process known as reflective PPG by placing the photo transmitter 112 and the photodetector 114 against the skin surface of an appendage of a subject (e.g., a finger, an earlobe, or any other suitable body part). The photo transmitter 112 emits a light that illuminates the blood inside a vein or artery that is adjacent to the skin surface and is absorbed by red hemoglobin in the blood. The volume of blood inside the vein or artery at a given time will periodically vary with each heartbeat, and the amount of light from the phototransmitter 112 that is absorbed by the blood is a function of the volume of blood in the vein or artery at a given time. Thus, the amount of light absorbed the blood periodically varies with each heartbeat. The photodetector 114 measures the amount of light (e.g., color and/or intensity) reflected back through the skin appendage and detects the changes in blood volume that correspond to blood flow of a heartbeat.

Similarly, as shown in FIG. 1B, the sensor can be configured as a transmissive optical sensor 310 that includes a photo transmitter or light source 312 (e.g., a light-emitting diode or “LED”) and a photodetector 314. The transmissive optical sensor 310 can be used to obtain a photoplethysmogram (“PPG”) using a process known as transmissive PPG by placing the photo transmitter 112 and the photodetector 114 against the skin surface on opposite sides of an appendage of a subject (e.g., a finger, an earlobe, or any other suitable body part). The photo transmitter 112 emits a light that illuminates the blood inside the veins or arteries of the appendage and is absorbed by red hemoglobin in the blood. The volume of blood inside the vein or artery at a given time will periodically vary with each heartbeat, and the amount of light from the phototransmitter 112 that is absorbed by the blood is a function of the volume of blood in the vein or artery at a given time. Thus, the amount of light absorbed the blood periodically varies with each heartbeat. The photodetector 114 measures the amount of light (e.g., color and/or intensity) transmitted through the appendage and detects the changes in blood volume that correspond to blood flow of a heartbeat.

As shown in FIG. 1C, the optical sensors 110 and 310 create a PPG waveform which is represented as amplitude versus time. The maximum amplitude of the PPG waveform corresponds to a systolic phase of a heartbeat (i.e., when the ventricles of the heart contract) and the minimum amplitude of the PPG waveform corresponds to the diastolic phase of the heartbeat (i.e., when the heart refills with blood). The amplitude of the PPG waveform is a measure of the volume of blood in the vein or artery and the period of the PPG waveform is a measure of heart rate. Thus, the PPG optical sensor can be used to detect blood flow and determine a measure of heart rate and/or cardiac cycle of an individual. The optical flow simulator 100 can be used to create a simulated optical signal that is substantially similar to an optical signal expected to be produced by blood flow as described above.

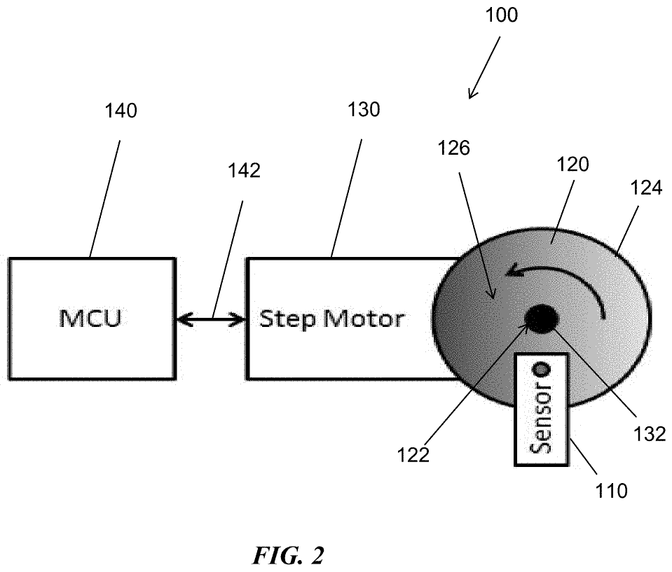

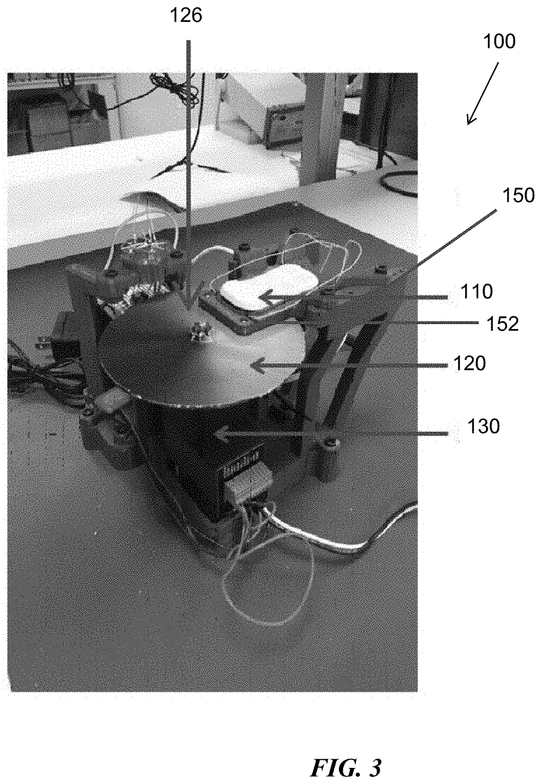

FIGS. 2 and 3 show an optical flow simulator device 100 according to some embodiments of the invention. The optical flow simulator 100 can include a disk 120, a stepper motor 130 connected to a microcontroller 140 by a wired or wireless connection 142, and a frame 150. Generally, the optical flow simulator 100 produces a simulated optical signal (e.g., color and intensity) for testing a reflective and/or transmissive optical sensor 110.

FIG. 2 shows a diagrammatic view of an optical flow simulator 100 according to some embodiments of the invention. As shown in FIG. 3, the optical flow simulator 100 can include an optional frame 150 which includes a platform 152. The platform 152 (FIG. 3) of the frame 150 can be used to support a reflective optical sensor 110 and positions the reflective optical sensor 110 at a predefined distance with respect to the optical surface of the disk 120. The frame 150 can be made of, for example, a metal material, a polymer material, or any other suitable material, or any combination thereof. While the frame 150 is shown as having one platform 152, the frame 150 can include a plurality of platforms for supporting a plurality of optical sensors adjacent to the disk 120. The bigger disk 120 can enable the configuration and testing of more sensors 110.

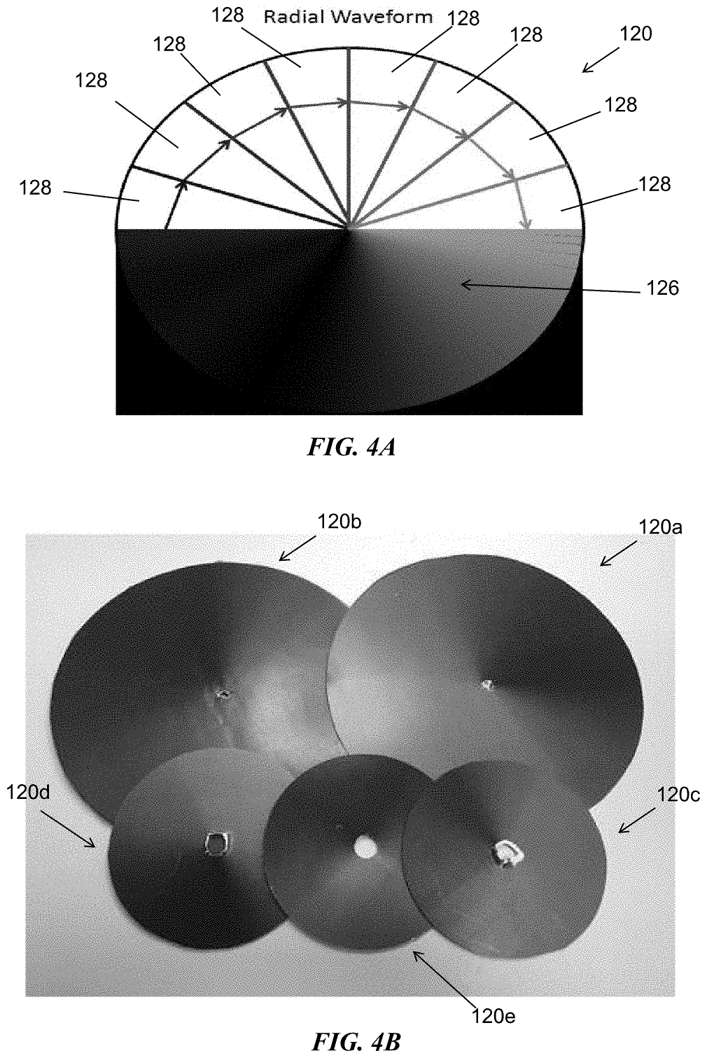

Generally referring to FIGS. 2 and 3, the disk 120 can include an optional central aperture 122, an outer edge 124, and a color distribution 126. The disk 120 can have a generally circular configuration, and can be made from, for example, a standard CD-ROM or DVD, a metal material, a polymer material, or any other suitable material, or any combination thereof. As best shown in FIG. 4A, the color distribution 126 varies along a path (e.g., that follows a track of the disk 120) and comprises a plurality of arc segments 128, with each of the plurality of arc segments 128 having a predefined color (e.g., green, yellow, blue, purple, red, or orange) or color variation. In accordance with some embodiments of the invention, each of the plurality of arc segments 128 can have substantially equal arc lengths and surface areas. In accordance with some embodiments of the invention, at least some of the arc segments 128 can have substantially different arc lengths and surface areas than other arc segments. In accordance with some embodiments of the invention, the width of the track or path can be in the range from the entire radial distance of disk to 1 mm or less. Preferably, the width of the track or path of the color distribution can be sufficiently wide to interact with optical sensor 110 to reflect the optical signal back to the optical sensor 110.

To form the color distribution 126 of the disk 120, the color distribution 126 can be printed or sprayed on a paper disk having substantially the same size and shape as the disk 120. The color distribution can be generated from a computerized image that matches the optical properties of the fluid to be monitored by the sensor 110. The paper disk can be coupled to or mounted on the upper surface of the disk 120, for example, via a permanent or removable adhesives or by clips or clamps. Alternatively, the paper disk can be removable from the disk 120 by, for example, a hook and loop fastener, or pressure clips. The color distribution 126 can be printed on the paper disk via an ink-jet printer, a laser-jet printer, or any other suitable mechanism. Alternatively, in some implementations, the color distribution 126 can be directly printed or sprayed on the surface or edge of disk 120.

The individual color of each of the plurality of arc segments 128 of the color distribution 126 (e.g., from dark green to light green, or dark red to light red) can be selected such that, when rotated at a predefined speed, the color distribution 126 produces an optical signal in the optical sensor that simulates the fluid flow to be monitored by PPG. As described herein, the reflective optical sensor 110 measures the amount of light absorbed by blood in an artery or vein when placed against an individual's skin. Similarly, when the reflective optical sensor 110 is positioned adjacent to the color distribution 126, as shown in FIGS. 1 and 2, the reflective optical sensor 110 measures the light reflected by each of the plurality of arc segments 128 as they move under the sensor 110. The amount of light reflected by each of the plurality of arc segments 128 is a function of the color of the arc segment, the amount of light absorbed by each arc segment and characteristics of the light produced by the sensor and directed toward the arc segment 128. As the disk 120 rotates, each of the plurality of arc segments 128 passes under the reflective optical sensor 110 (which is held substantially stationary by the frame 150), and the reflective optical sensor 110 measures the amount of light reflected by the color distribution 126. By selectively controlling the individual color of each of the plurality arc segments 128, the color distribution 126 controls the amplitude of the reflected optical signal to simulate the light reflected by the fluid flow (e.g., blood flow). In addition, the period (or frequency) of the optical signal can be controlled by selecting a predefined rotational speed of the disk 120. In this manner, rotation of the disk 120 produces an optical signal that is substantially similar to a real PPG waveform.

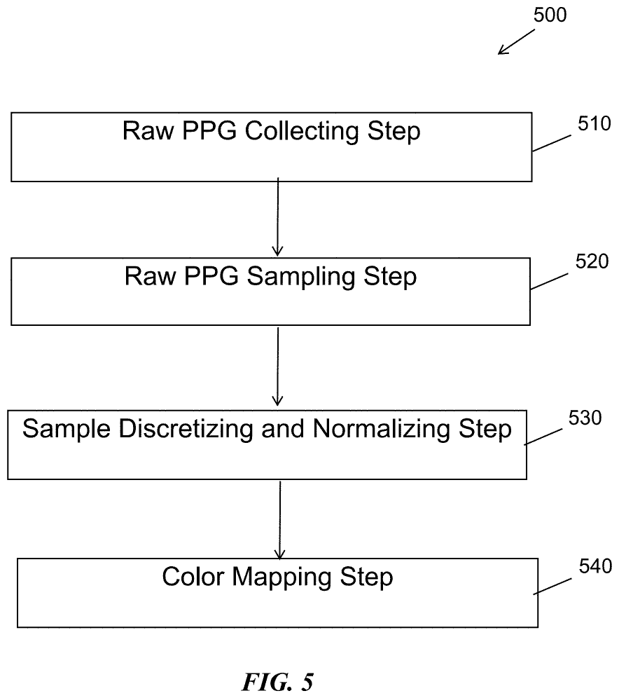

To control the amplitude of the optical signal produced by the disk 120, the color of each of the plurality of arc segments 128 of the color distribution 126 is selected according to an algorithm 500, which is illustrated by the flow diagram of FIG. 5. The algorithm 500 can include a raw PPG waveform collecting step 510, a PPG sampling step 520, a sample discretizing and normalizing step 530, and a color mapping step 540.

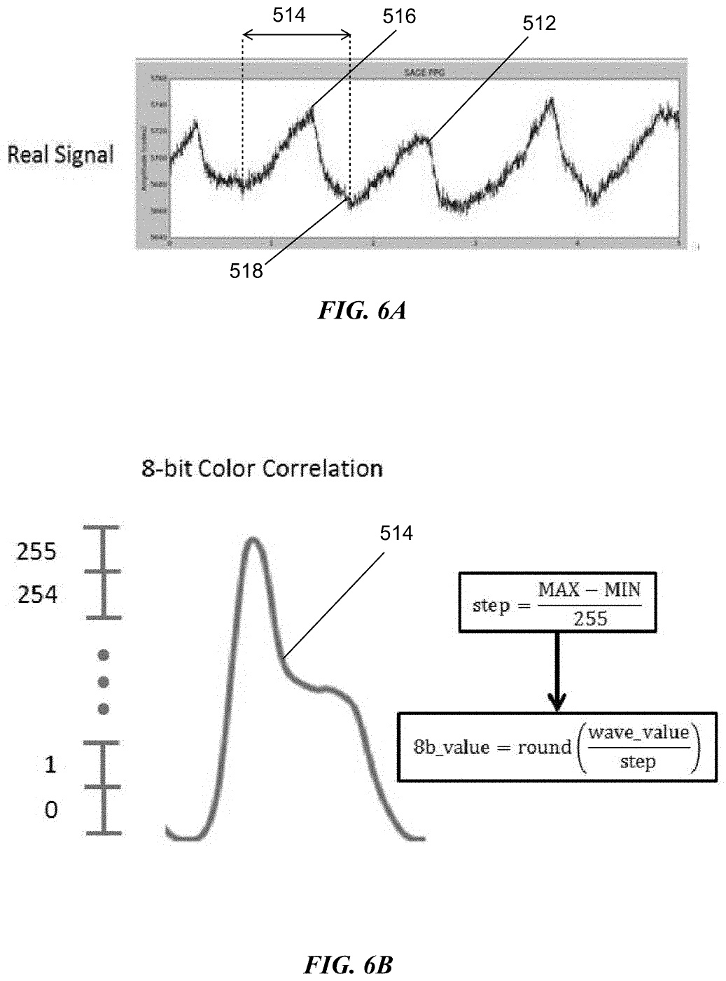

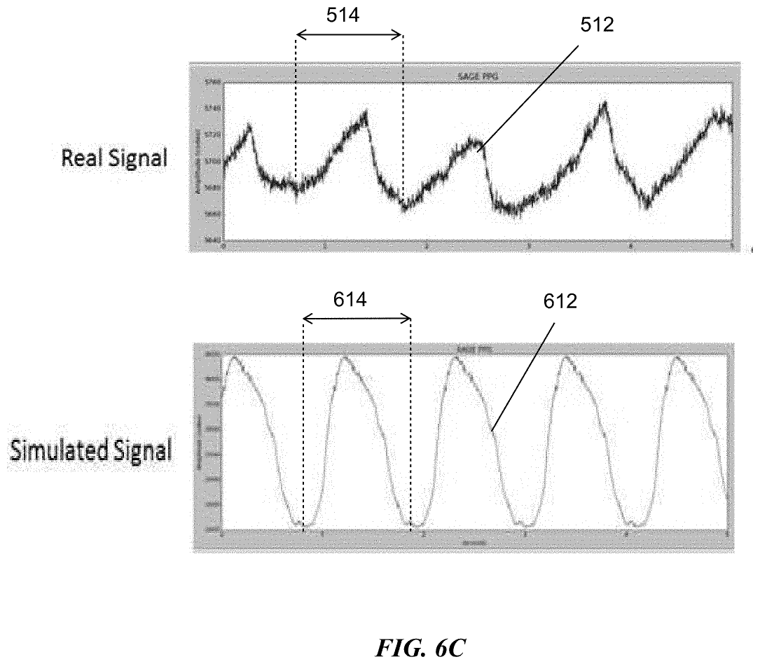

During the raw PPG waveform collecting step 510, a raw PPG waveform 512 (FIG. 6A) is collected from an individual via, for example, the reflective optical sensor 110, or any other suitable mechanism (e.g., a transmissive optical sensor). For example, because blood absorbs more light than the surrounding tissue, increased blood volume which results during a heartbeat causing blood vessels to expand can be indicated by a decrease in the detected light intensity. The raw PPG waveform 512 can also be obtained from an archive of previously obtained PPG waveforms, or artificially generated via a computer model, or any other suitable mechanism, or any combination thereof. As shown in FIG. 6A, the raw PPG waveform 512 is a function of amplitude versus time. As described above, the amplitude (e.g., sensed light intensity) of the raw PPG waveform 512 corresponds (e.g., inversely) to the volume of blood in the veil or artery at a given time and each period of the raw PPG waveform 512 (e.g., sample period 514 shown in FIG. 6A) corresponds to one heartbeat. Thus, the light intensity decreases during a heartbeat pulse and increases between pulse beats.

During the sampling step 520, a sample period 514 is selected from the raw PPG waveform 512. During the sample discretizing and normalizing step 530, the sample period 514 is separated into discrete segments (e.g., heartbeat time segment). As shown in FIG. 6B, the sample period 514 is separated into 256 discrete segments (although, more or less segments could be used); the same number as the number of arc segments in the plurality of arc segments 128 that forms the color distribution 126 (FIG. 4B). To normalize the amplitude values of the sample period 514, the amplitude of each discrete segment can be adjusted to a common scale (e.g., an 8 bit scale) so that the minimum value for any given discrete segment is 0 and the maximum value is 255. To accomplish this normalization, a step value is calculated according to equation (1) below by measuring the maximum measured amplitude 516 (“MAX”) and the minimum measured amplitude 518 (“MIN”) of the sample period 514:

step value=MAX−MIN/255 (1)

Next, each of the 256 discrete segments of the period 514 is assigned an eight-bit value ranging between 0 and 255 according to equation (2) below by dividing the measured amplitude of the segment by the step value and rounding to the nearest whole integer:

eightbit value=amplitude/step value (2)

Each eight-bit value, which ranges between 0 and 255, can also be represented as 8 individual bits (0, 1, 2, 3, 4, 5, 6, 7), with each bit having a value of 0 or 1. For higher resolution (e.g., 16 bit) or lower resolution (e.g., 4 bit), the divisor in equation (1) can be increased (e.g., 65536) or decreased (e.g., 16), respectively.

During the color mapping step 540, the eight-bit value of each of the 256 discrete segments can be converted to a color value according to any known color system model (e.g., RGB (Red, Green, Blue color space), HSV (Hue, Saturation, Value color space), HLS (Hue, Luminance, Saturation color space), or CMYK (Cyan, Magenta, Yellow and Key-BlacK color space)). Alternatively, the color mapping could simply map the amplitude to the intensity, hue, and/or saturation space of the detected signal for a single color (e.g. monochrome) or a plurality of colors. Thus, the eight-bit values can be converted into 256 (or more or less) different possible color values, which are then assigned to each of the 256 discrete segments. Using these color values, the individual color of each of the plurality of arc segments 128 (FIG. 4B) is selected.

Because one period of the raw PPG waveform 512 (i.e., one heartbeat) was derived from the raw PPG waveform sampling step 510, the optical signal simulates one heartbeat for each revolution of the disk 120. As shown in FIG. 6C, an optical signal 612 produced by the color distribution 126 has an amplitude that is substantially similar to the raw PPG waveform 512. Further, period 614 of the optical signal 612 is produced by one full revolution of the disk 120 and simulates one full heartbeat.

Alternatively, while the sample period 514 is shown in FIGS. 6A and 6C as encompassing a single period of the raw PPG waveform 512 (i.e. one heartbeat), any number of periods of the raw PPG waveform 512 (i.e., two or more heartbeats) can be selected as the sample period 514. In such implementations, one revolution of the disk 120 can be configured to produce an optical signal that simulates two or more full heartbeats.

While the sample period 514 is described as being discretized into 256 segments during the sample discretizing and normalizing step 530, the sample period 514 can also be separated into more or less discrete segments, for example, 65,536 or 16 discrete segments. In such implementations, each of the 65,536 discrete segments of the sample period 514 can be assigned a sixteen-bit value during step 530. During the color mapping step 540, each discrete segment can be assigned a color value using any color model representation. In this configuration, the plurality of arc segments 128 includes 65,536 arc segments each having a color assigned by the color mapping step 540.

Referring to FIGS. 2 and 3, the stepper motor 130 includes a spindle 132 and can be energized to rotate the disk 120 in order to produce the predefined optical signal. While the stepper motor 130 shown in FIG. 2 can be a brushless direct-current (“DC”) electric motor, any other suitable mechanism for rotating the disk 120 can be used (e.g., a brushed DC electric motor, an internal combustion engine, etc.). The disk 120 can be mounted to the spindle 132 of the stepper motor via the central aperture 122 of the disk 120 such that rotation of the spindle 132 causes rotation of the disk 120. The spindle 132 can be secured within the central aperture 122 of the disk 120 by various mechanisms, such as, for example, a friction fit, a snap fit, a press fit, a set screw, a locking collar, a welded connection, an adhesive connection, or any other suitable mechanism, or any combination thereof. Alternatively, the spindle 132 can be coupled to a bottom surface of the disk 120 via an adhesive connection, a welded connection, a magnetic connection, a hoop and loop fastener, or the like, or any combination thereof.

To rotate the disk 120 at a predefined speed, the disk 120 (FIG. 2) can be operatively coupled to the stepper motor 130 (e.g., either directly or by a transmission such as gears and/or belts) and controls the rotational speed of the spindle 132 and the disk 120. The microcontroller 140 can be operatively connected to the stepper motor 130 by a wired or wireless connection 142. The software that is executed by microcontroller 140 can include one or more modules that drive a motor control circuit that cause the stepper motor to rotate at a predefined step rate, for example, 200 steps per second at 200 steps per revolution provides 60 revolutions per minute rotation. For example, using the microcontroller 140, the predefined rotational speed of the disk 120 can be set to 10 revolutions per minute (RPM), 20 RPM, 60 RPM, 100 RPM, 200 RPM, etc. As described above, the period (or frequency) of the simulated optical signal produced by rotation of the disk 120 is a function of the rotational speed of the disk 120, and the configuration of the color distribution image 126 that covers one full revolution of the disk 120 (e.g., number of full heartbeats simulated by each revolution of the disk). Thus, the predefined rotational speed of the disk 120 controlled by the stepper motor 130 and the microcontroller 140 can be used to simulate a predefined heart rate. For example, for a single beat per revolution color distribution image 126, a predefined rotational speed of 60 RPM simulates a heart rate of 60 beats per minute (BPM).

In some implementations, the position of the reflective optical sensor 110 can be varied relative to the outer edge 124 of the disk 120 to increase or decrease the signal-to-noise ratio of the simulated optical signal. As the position of the reflective optical sensor 110 moves relative to the outer edge 124 towards the central aperture 122 (and/or vice versa), the color distribution 126 of the disk 120 is the same at each location on the disk 120 between the outer edge 124 and central aperture 122. Thus, the period of the optical signal will be the same regardless of the relative position of the reflective optical sensor 110 between the outer edge 124 and the central aperture 122. However, the signal-to-noise ratio of the simulated optical signal is a function of the relative position of the reflective optical sensor 110. When the reflective optical sensor 110 is positioned closer to the outer edge 124, the light emitted by the photo transmitter 112 illuminates an effectively larger area than when the reflective optical sensor 110 is positioned closer to the central aperture 122. Thus, positioning the reflective optical sensor 110 closer to the outer edge 124 decreases the influence of other portions of the color distribution 126 (i.e., noise) and increases the signal-to-noise ratio of the simulated optical signal.

Because (i) the color distribution 126 of the disk 120 controls the amplitude of the optical signal and (ii) the stepper motor 130 and microcontroller 140 rotate the disk 120 at a predefined rotational speed to control the period of the optical signal, the optical flow simulator 100 can be used to test the functioning of the reflective optical sensor 110 in a variety of ways. For instance, if the predefined rotational speed of the disk 120 is set to 60 RPM, and the reflective optical sensor 110 measures a simulated heart rate that is not substantially equal to 60 BPM, a user is alerted to an error in the reflective optical sensor 110 design or manufacture. Alternatively, as shown in FIG. 4B, the disk 120 can be easily and quickly substituted with another disk (e.g., disk 120a, disk 120b, disk 120c, disk 120d, and/or disk 120e) with a different color distribution selected according to algorithm 500 to simulate a different PPG signal.

In yet another example, the stepper motor 130 and the microcontroller 140 can be used to rotate the disk 120 at a plurality of predefined speeds in succession to test the sensitivity of the reflective optical sensor 110 to changes in heart rate. The predefined rotational speed of the disk 120 can be slowly increased from, for example, 60 RPM to 180 RPM in order to simulate an increase in heart rate from 60 BPM for 180 BPM. Alternatively, the microcontroller 140 can be used change the rotational speed of the disk 120 from a first predefined rotational speed to a second predefined rotational speed for a very short (i.e., substantially instantaneous) period of time, and then immediately return the disk 120 to the first predefined rotational speed in order to test the reflective optical sensor 110's ability to detect slight variances in heart rate.

Advantageously, the optical flow simulator 100 allows a user to test an optical sensor's ability to detect unhealthy heart conditions (e.g., arrhythmia) without having to apply the sensor to a patient with said unhealthy heart condition, thus increasing testing efficiency while decreasing testing costs.

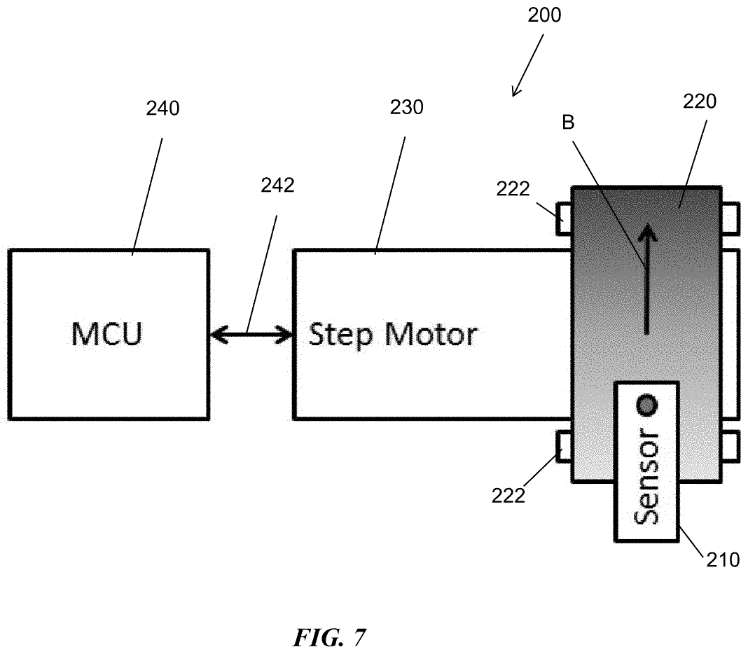

Referring to FIG. 7, an optical flow simulator 200 is similar to the optical flow simulator 100 in that it includes a stepper motor 230 connected to a microcontroller 240 by a wired or wireless connection 242, and a frame (not shown). The optical flow simulator 200 produces an optical signal for testing a reflective optical sensor 210 that is the same as or similar to reflective optical sensor 110. The optical flow simulator 200 differs from optical flow simulator 100 in that it includes a belt 220 (rather than the disk 120 of the optical flow simulator 100) that can be extended over a plurality of rollers 222. The belt 220 can have a color distribution 226 that is similar to the color distribution 126 in that it includes a plurality of linear segments (not shown), each having a color that is selected to control the amplitude of the simulated optical signal. The color distribution 226 differs from the color distribution 126, however, in that instead of varying along a curved path, the color distribution 226 varies along a linear path on the belt. The individual color for each of the plurality of linear segments can be selected using an algorithm that is the same as or similar to the algorithm 500 of the optical flow simulator 100.

The stepper motor 230 and the microcontroller 240 can be similar to the stepper motor 130 and the microcontroller 140 of the optical flow sensor 100. The stepper motor 230 differs, however, in that it rotates at least one of the plurality of rollers 222 over which the belt 220 is extended, thereby moving the belt 220 along arrow B. Just as the rotational speed of the disk 120 controls the period (or frequency) of the simulated optical signal produced by the optical flow simulator 100, a linear speed of the belt 220 controls the period (or frequency) of the simulated optical signal produced by the optical flow simulator 200. In accordance with some embodiments of the invention, the rotational speed of the stepper motor 230 and the diameter of the roller determine the speed the belt and depending on the configuration of the color distribution (e.g., how many heart beats are simulated by a full revolution of the belt), the heart rate can be simulated. For example, for one heart beat per revolution of the belt (e.g, of length L) on a drive roller of diameter D, the stepper motor 230 would need to rotated at a speed of (60×L)/(π×D) in revolutions per minute.

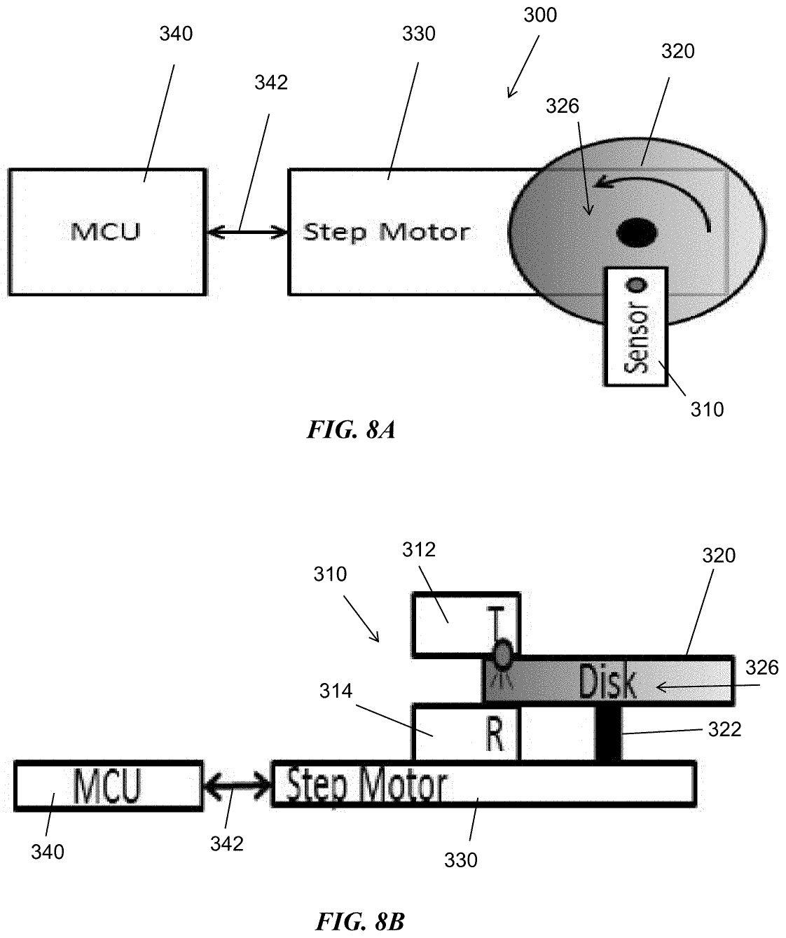

Referring to FIGS. 8A and 8B, an optical flow simulator 300 includes a disk 320, a stepper motor 330 connected to a microcontroller 340 by a wired or wireless connection 342, and a frame (not shown). The optical flow simulator 300 is similar to the optical flow simulators 100, 200 in that it produces a simulated optical signal, but differs in that the simulated optical signal is for testing a transmissive optical sensor 310. Referring to FIGS. 3B and 8B, the transmissive optical sensor 310 is similar to the reflective optical sensor 110 in that it includes a photo transmitter 312 and a photodetector 314 and measures a heart rate and/or cardiac cycle of an individual. The transmissive optical sensor 310 varies, however, in that the photodetector 314 measures the amount of light from the photo transmitter that passes through the appendage of the individual.

The stepper motor 330 and the microcontroller 340 can be the same as the stepper motor 130 and the microcontroller 140 of the optical flow sensor 100. The disk 320 is similar to the disk 120 of the optical flow sensor 100 but differs in that it is made from a semi-translucent material and has a light transmissivity distribution 326. The light transmissivity distribution 326 is similar to the color distribution 126 of the disk 120 in that it varies along a circular path. Because the optical flow simulator 300 is used to test the transmissive optical sensor 310, as opposed to the reflective optical sensor 110, the light transmissivity distribution 326 is selected to vary the amount of light that passes through the disk 320. Thus, the light transmissivity distribution 326 controls the amplitude of the optical signal produced by the optical flow simulator 300 in the same or similar manner as the color distribution 126 of the optical flow simulator 100. Likewise, the light transmissivity distribution 326 is selected using an algorithm that is the same or similar to the algorithm 500 used to select the color distribution 126 of the optical flow simulator 100.

In some implementations, an optical flow simulator that is similar to the optical flow simulator 100 includes a disk, a stepper motor, a microcontroller, and an optical frame. The disk includes a color distribution (or a light transmissivity distribution) of the disk can be positioned on a side surface of the disk defined by the circumference of the disk (not shown). In such implementations, the color distribution is similar to the color distribution 126 in that it produces an optical signal, but differs in that it comprises a plurality of linear segments, each of which having an individual color. The reflective optical sensor is positioned directly adjacent to the side surface of the disk. As the disk rotates via the stepper motor and microcontroller in the same or similar manner as the disk 120 described above, a different one of the plurality of linear segments of the color distribution passes adjacent to the reflective optical sensor. Thus, the color distribution creates an optical signal that is the same as or similar to the optical signal of the optical flow simulator 100.

While the present invention has been described with reference to one or more particular embodiments, those skilled in the art will recognize that many changes may be made thereto without departing from the spirit and scope of the present invention. Each of these embodiments and obvious variations thereof fall within the spirit and scope of the invention. Additional embodiments according to aspects of the present invention can combine any number of features from any of the embodiments described herein.

1. An optical flow simulator, comprising:
a disk having a color distribution varied along a path on the disk;
a stepper motor configured to rotate a spindle, the spindle being coupled to the disk;
a microcontroller for controlling the stepper motor such that the spindle and the disk rotate at a predefined rotational speed; and
wherein the color distribution produces an optical signal responsive to rotation of the disk at the predefined rotational speed.

2. The optical flow simulator of claim 1, further comprising a frame having a platform for positioning at least one reflective optical sensor directly adjacent to the color distribution of the disk such that the least one reflective optical sensor can measure the simulated optical signal.

3. The optical flow simulator of claim 1, wherein the color distribution of the disk is selected according to an algorithm such that the simulated optical signal is substantially similar to a measured optical signal.

4. The optical flow simulator of claim 3, wherein the color distribution of the disk comprises a plurality of arc segments, each of the plurality of arc segments having an individual color that is selected according to the algorithm.

5. The optical flow simulator of 3, wherein the measured optical signal is a Photoplethysmogram (PPG) waveform obtained from an individual, wherein each period of the PPG waveform measures one heartbeat of the individual.

6. The optical flow simulator of claim 7, wherein the simulated optical signal simulates one heartbeat from the PPG waveform for each full revolution of the disk.

7. The optical flow simulator of claim 7, wherein (i) at least one reflective optical sensor is fixed directly adjacent to the color distribution and (ii) the predefined rotational speed is selected such that a period of the simulated optical signal simulates a heart rate.

8. The optical flow simulator of claim 7, wherein at least one reflective optical sensor is selectively positioned directly adjacent to the color distribution relative to an outer edge thereof such that a period of the simulated optical signal simulates a heart rate.

9. An optical flow simulator, comprising:
a belt having a color distribution linearly varied along a path, the belt being fitted over a plurality of rollers;
a stepper motor configured to rotate a spindle, the spindle being configured to rotate at least one of the plurality of rollers to move the belt;
a microcontroller for controlling the stepper motor such that the belt moves at a predefined speed; and
wherein the color distribution produces a simulated optical signal responsive to movement of the belt at the predefined speed.

10. The optical flow simulator of 9, further comprising a frame having a platform for positioning at least one reflective optical sensor directly adjacent to the color distribution of the belt such that the least one reflective optical sensor can measure the simulated optical signal.

11. The optical flow simulator of claim 9, wherein the color distribution of the belt is selected according to an algorithm such that the simulated optical signal is substantially similar to a measured optical signal.

12. The optical flow simulator of claim 11, wherein the color distribution of the belt comprises a plurality of arc segments, each of the plurality of arc segments having an individual color that is selected according to the algorithm.

13. The optical flow simulator of 11, wherein for each revolution of the belt at the predefined speed, the simulated optical signal is substantially similar to one period of the physically measured optical signal.

14. The optical flow simulator of 11, wherein the predefined speed is selected such that a frequency of the simulated optical signal measured by the at least one reflective optical sensor is substantially similar to a frequency of the physically measured optical signal.

15. An optical flow simulator, comprising:
a semi-translucent disk having an light transmissivity distribution varied along a path on the semi-translucent disk;
a stepper motor configured to rotate a spindle, the spindle being coupled to the semi-translucent disk;
a microcontroller for controlling the stepper motor such that the spindle and the disk rotate at a predefined rotational speed; and
wherein the light transmissivity distribution of the semi-translucent disk produces a simulated optical signal responsive to rotation of the disk at the predefined rotational speed.

16. The optical flow simulator of 15, further comprising a frame having a first platform for positioning a photo transmitter of a transmissive optical sensor directly adjacent to a first surface of the semi-translucent disk and a second platform for positioning a photo-receiver of the transmissive optical sensor directly adjacent to a second surface of the semi-translucent disk such that the transmissive optical sensor can measure the simulated optical signal.

17. The optical flow simulator of 15, wherein the light transmissivity distribution is selected according to an algorithm such that the simulated optical signal corresponds to a physically measured optical signal.

18. The optical flow simulator of claim 17, wherein the light transmissivity distribution of the semi-translucent disk comprises a plurality of segments, each of the plurality of segments having a light transmissivity that is selected according to the algorithm.

19. The optical flow simulator of 17, wherein for each revolution of the semi-translucent disk at the predefined rotational speed, the simulated optical signal is substantially similar to one period of the physically measured optical signal.

20. The optical flow simulator of 17, wherein (i) at least one transmissive optical sensor is positioned at a fixed location directly adjacent to the light transmissivity distribution of the disk and (ii) the predefined rotational speed is selected such that a frequency of the simulated optical signal measured by the at least one transmissive optical sensor is substantially similar to a frequency of the physically measured optical signal.

21. The optical flow simulator of 17, wherein at least one transmissive optical sensor is selectively positioned directly adjacent to the light transmissivity distribution of the semi-translucent disk relative to an outer edge thereof such that a frequency of the simulated optical signal measured by the at least one transmissive optical sensor is substantially similar to a frequency of the physically measured optical signal.

22-25. (canceled)
